(19) United States
(12) Patent Application Publication
(10) Pub. No.: US 2003/0204101 A1
(43) Pub. Date: Oct. 30, 2003

(54) Direct epoxidation process using improved catalyst composition

(75) Inventors: Jennifer D. Jewson, Boyertown, PA (US); C. Andrew Jones, Newtown Square, PA (US); Ralph M. Dessau, Edison, NJ (US)

Correspondence Address: LYONDELL CHEMICAL COMPANY, 3801 WEST CHESTER PIKE, NEWTOWN SQUARE, PA, 19073, US

(73) Assignee: ARCO CHEMICAL TECHNOLOGY, L.P.

(21) Appl. No.: 10/435,175

(22) Filed: May 9, 2003

Related U.S. Application Data
(62) Division of application No. 09/507,842, filed on Feb. 22, 2000, now abandoned.

(51) Int. Cl. C07D 301/06; B01J 29/06

(52) U.S. Cl. 549/533; 502/66

(57) ABSTRACT

Highly selective and productive epoxidation catalysts are prepared by combining a titanium zeolite, palladium, and a gold promoter. The resulting materials are useful catalysts for transforming olefins to epoxides in the reaction of an olefin, hydrogen, and oxygen.

### FIELD OF THE INVENTION

This invention relates to an epoxidation process using an improved palladium-titanosilicate catalyst and a method of producing the improved catalyst. The catalyst is a palladium-titanosilicate that contains a gold promoter. Surprisingly, the promoted catalyst shows improved selectivity and productivity in the epoxidation of olefins with oxygen and hydrogen compared to a palladium-titanosilicate without a gold promoter.

### BACKGROUND OF THE INVENTION

Many different methods for the preparation of epoxides have been developed. Generally, epoxides are formed by the reaction of an olefin with an oxidizing agent in the presence of a catalyst. The production of propylene oxide from propylene and an organic hydroperoxide oxidizing agent, such as ethyl benzene hydroperoxide or tert-butyl hydroperoxide, is commercially practiced technology. This process is performed in the presence of a solubilized molybdenum catalyst, see U.S. Pat. No. 3,351,635, or a heterogeneous titania on silica catalyst, see U.S. Pat. No. 4,367,342. Hydrogen peroxide is another oxidizing agent useful for the preparation of epoxides. Olefin epoxidation using hydrogen peroxide and a titanium silicate zeolite is demonstrated in U.S. Pat. No. 4,833,260. One disadvantage of both of these processes is the need to pre-form the oxidizing agent prior to reaction with olefin.

Another commercially practiced technology is the direct epoxidation of ethylene to ethylene oxide by reaction with oxygen over a silver catalyst. Unfortunately, the silver catalyst has not proved very useful in epoxidation of higher olefins. Therefore, much current research has focused on the direct epoxidation of higher olefins with oxygen and hydrogen in the presence of a catalyst. In this process, it is believed that oxygen and hydrogen react in situ to form an oxidizing agent. Thus, development of an efficient process (and catalyst) promises less expensive technology compared to the commercial technologies that employ pre-formed oxidizing agents.

Many different catalysts have been proposed for use in the direct epoxidation of higher olefins. For example, JP 4-352771 discloses the epoxidation of propylene oxide from the reaction of propylene, oxygen, and hydrogen using a catalyst containing a Group VIII metal such as palladium on a crystalline titanosilicate. Other examples include gold supported on titanium oxide, see for example U.S. Pat. No. 5,623,090, and gold supported on titanosilicates, see for example PCT Intl. Appl. WO 98/00413. Although the use of promoters is disclosed in PCT Intl. Appl. WO 98/00413, a palladium promoter is specifically excluded.

U.S. Pat. No. 5,859,265 discloses a catalyst in which a platinum metal, selected from Ru, Rh, Pd, Os, Ir and Pt, is supported on a titanium or vanadium silicalite. Additionally, it is disclosed that the catalyst may also contain additional elements, including Fe, Co, Ni, Re, Ag, or Au. However, the examples of the patent show only the preparation and use of a palladium-impregnated titanosilicate catalyst and the patent offers no reason for the addition of the other elements or a method of incorporating the additional elements.

One disadvantage of the described direct epoxidation catalysts is that they all show either less than optimal selectivity or productivity. As with any chemical process, it is desirable to attain still further improvements in the direct epoxidation methods and catalysts. In particular, increasing the selectivity to epoxide, the productivity of the catalyst, and extending the useful life of the catalyst would significantly enhance the commercial potential of such methods.

We have discovered an effective, convenient epoxidation catalyst that gives higher selectivity to epoxide and higher productivity compared to comparable palladium-titanosilicate catalysts.

### SUMMARY OF THE INVENTION

The invention is an olefin epoxidation process that comprises reacting olefin, oxygen, and hydrogen in the presence of a catalyst comprising a titanium zeolite, palladium, and a gold promoter. We surprisingly found that catalysts produced with the addition of gold promoter give significantly higher selectivity to epoxide and have higher productivity compared to catalysts without the gold promoter.

### DETAILED DESCRIPTION OF THE INVENTION

The process of the invention employs a catalyst that comprises a titanium zeolite, palladium, and a gold promoter. Suitable titanium zeolites are those crystalline materials having a porous molecular sieve structure with titanium atoms substituted in the framework. The choice of titanium zeolite employed will depend upon a number of factors, including the size and shape of the olefin to be epoxidized. For example, it is preferred to use a relatively small pore titanium zeolite such as a titanium silicalite if the olefin is a lower aliphatic olefin such as ethylene, propylene, or 1-butene. Where the olefin is propylene, the use of a TS-1 titanium silicalite is especially advantageous. For a bulky olefin such as cyclohexene, a larger pore titanium zeolite such as a titanium zeolite having a structure isomorphous with zeolite beta may be preferred.

Titanium zeolites comprise the class of zeolitic substances wherein titanium atoms are substituted for a portion of the silicon atoms in the lattice framework of a molecular sieve. Such substances are well known in the art.

Particularly preferred titanium zeolites include the class of molecular sieves commonly referred to as titanium silicalites, particularly “TS-1” (having an MFI topology analogous to that of the ZSM-5 aluminosilicate zeolites), “TS-2” (having an MEL topology analogous to that of the ZSM-11 aluminosilicate zeolites), and “TS-3” (as described in Belgian Pat. No. 1,001,038). Titanium-containing molecular sieves having framework structures isomorphous to zeolite beta, mordenite, ZSM-48, ZSM-12, and MCM-41 are also suitable for use. The titanium zeolites preferably contain no elements other than titanium, silicon, and oxygen in the lattice framework, although minor amounts of boron, iron, aluminum, sodium, potassium, copper and the like may be present.

Preferred titanium zeolites will generally have a composition corresponding to the following empirical formula xTiO₂ (1−x)SiO₂ where x is between 0.0001 and 0.5000. More preferably, the value of x is from 0.01 to 0.125. The molar ratio of Si:Ti in the lattice framework of the zeolite is advantageously from 9.5:1 to 99:1 (most preferably from 9.5:1 to 60:1). The use of relatively titanium-rich zeolites may also be desirable.

The catalyst employed in the process of the invention also contains palladium. The typical amount of palladium present in the catalyst will be in the range of from about 0.01 to 20 weight percent, preferably 0.01 to 5 weight percent. The manner in which the palladium is incorporated into the catalyst is not considered to be particularly critical. For example, the palladium may be supported on the zeolite by impregnation or the like or first supported on another substance such as silica, alumina, activated carbon or the like and then physically mixed with the zeolite. Alternatively, the palladium can be incorporated into the zeolite by ion-exchange with, for example, Pd tetraamine chloride.

There are no particular restrictions regarding the choice of palladium compound used as the source of palladium. For example, suitable compounds include the nitrates, sulfates, halides (e.g., chlorides, bromides), carboxylates (e.g. acetate), and amine complexes of palladium. Similarly, the oxidation state of the palladium is not considered critical. The palladium may be in an oxidation state anywhere from 0 to +4 or any combination of such oxidation states. To achieve the desired oxidation state or combination of oxidation states, the palladium compound may be fully or partially pre-reduced after addition to the catalyst. Satisfactory catalytic performance can, however, be attained without any pre-reduction. To achieve the active state of palladium, the catalyst may undergo pretreatment such as thermal treatment in nitrogen, vacuum, hydrogen, or air.

The catalyst used in the process of the invention also contains a gold promoter. The typical amount of gold present in the catalyst will be in the range of from about 0.01 to 10 weight percent, preferably 0.01 to 2 weight percent. While the choice of gold compound used as the gold source in the catalyst is not critical, suitable compounds include gold halides (e.g., chlorides, bromides, iodides), cyanides, and sulfides. Although the gold may be added to the titanium zeolite before, during, or after palladium addition, it is preferred to add the gold promoter at the same time that palladium is introduced. Any suitable method can be used for the incorporation of gold into the catalyst. As with palladium addition, the gold may be supported on the zeolite by impregnation or the like or first supported on another substance such as silica, alumina, activated carbon or the like and then physically mixed with the zeolite. Incipient wetness techniques may also be used to incorporate the gold promoter. In addition, the gold may be supported by a deposition-precipitation method in which gold hydroxide is deposited and precipitated on the surface of the titanium zeolite by controlling the pH and temperature of the aqueous gold solution (as described in U.S. Pat. No. 5,623,090).

After palladium and gold incorporation, the catalyst is recovered. Suitable catalyst recovery methods include filtration and washing, rotary evaporation and the like. The catalyst is typically dried at a temperature greater than about 50° C. prior to use in epoxidation. The drying temperature is preferably from about 50° C. to about 200° C. The catalyst may additionally comprise a binder or the like and may be molded, spray dried, shaped or extruded into any desired form prior to use In epoxidation.

The epoxidation process of the invention comprises contacting an olefin, oxygen, and hydrogen in the presence of the palladium/gold/titanium zeolite catalyst. Suitable olefins include any olefin having at least one carbon-carbon double bond, and generally from 2 to 60 carbon atoms. Preferably the olefin is an acyclic alkene of from 2 to 30 carbon atoms; the process of the invention is particularly suitable for epoxidizing C₂-C₆ olefins. More than one double bond may be present, as in a diene or triene for example. The olefin may be a hydrocarbon (i.e., contain only carbon and hydrogen atoms) or may contain functional groups such as halide, carboxyl, hydroxyl, ether, carbonyl, cyano, or nitro groups, or the like. The process of the invention is especially useful for converting propylene to propylene oxide.

Epoxidation according to the invention is carried out at a temperature effective to achieve the desired olefin epoxidation, preferably at temperatures in the range of 0-250° C., more preferably, 20-100° C. The molar ratio of hydrogen to oxygen can usually be varied in the range of H₂:O₂=1:10 to 5:1 and is especially favorable at 1:5 to 2:1. The molar ratio of oxygen to olefin is usually 1:1 to 1:20, and preferably 1:1.5 to 1:10. Relatively high oxygen to olefin molar ratios (e.g., 1:1 to 1:3) may be advantageous for certain olefins. A carrier gas may also be used in the epoxidation process. As the carrier gas, any desired inert gas can be used. The molar ratio of olefin to carrier gas is then usually in the range of 100:1 to 1:10 and especially 20:1 to 1:10.

As the inert gas carrier, noble gases such as helium, neon, and argon are suitable in addition to nitrogen and carbon dioxide. Saturated hydrocarbons with 1-8, especially 1-6, and preferably with 1-4 carbon atoms, e.g., methane, ethane, propane, and n-butane, are also suitable. Nitrogen and saturated C₁-C₄ hydrocarbons are the preferred inert carrier gases. Mixtures of the listed inert carrier gases can also be used.

Specifically in the epoxidation of propylene according to the invention, propane can be supplied in such a way that, in the presence of an appropriate excess of carrier gas, the explosive limits of mixtures of propylene, propane, hydrogen, and oxygen are safely avoided and thus no explosive mixture can form in the reactor or in the feed and discharge lines.

The amount of catalyst used may be determined on the basis of the molar ratio of the titanium contained in the titanium zeolite to the olefin that is supplied per unit time. Typically, sufficient catalyst is present to provide a titanium/olefin feed ratio of from 0.0001 to 0.1 hour. The time required for the epoxidation may be determined on the basis of the gas hourly space velocity, i.e., the total volume of olefin, hydrogen, oxygen and carrier gas(es) per unit hour per unit of catalyst volume (abbreviated GHSV). A GHSV in the range of 10 to 10,000 hr⁻¹ is typically satisfactory.

Depending on the olefin to be reacted, the epoxidation according to the invention can be carried out in the liquid phase, the gas phase, or in the supercritical phase. When a liquid reaction medium is used, the catalyst is preferably in the form of a suspension or fixed-bed. The process may be performed using a continuous flow, semi-batch or batch mode of operation.

If epoxidation is carried out in the liquid phase, it is advantageous to work at a pressure of 1-100 bars and in the presence of one or more solvents. Suitable solvents include, but are not limited to, lower aliphatic alcohols such as methanol, ethanol, isopropanol, and tert-butanol, or mixtures thereof, and water. Fluorinated alcohols can be used. It is also possible to use mixtures of the cited alcohols with water.

The following examples merely illustrate the invention. Those skilled in the art will recognize many variations that are within the spirit of the invention and scope of the claims.

### EXAMPLE 1

Preparation of Pd/Au/TS-1 Catalyst

TS-1 can be made according to any known literature procedure. See, for example, U.S. Pat. No. 4,410,501, DiRenzo, et. al., Microporous Materials (1997), Vol. 10, 283, or Edler, et. al., J. Chem. Soc., Chem. Comm. (1995), 155. The TS-1 is calcined at 550° C. for 4 hours before use.

The pre-calcined TS-1 (20 g), [Pd (NH₃)₄] (NO₃)₂ (2.06 g of a 5 weight percent Pd solution in water), AuCl₃ (0.0317 g), and distilled water (80 g) are placed in a 250-mL single-neck round-bottom flask forming a pale white mixture. The flask is connected to a 15-inch cold water condenser and then blanketed with nitrogen at a 150 cc/min flow rate. The flask is inserted into an oil bath at 80° C. and the reaction slurry is stirred. After stirring for 24 hours, the slurry is transferred to a roto-vap and the water is removed by roto-evaporation under vacuum at 50° C. The solid catalyst is then dried at 60° C. in a vacuum oven for 24 hours. Measured Pd loading of the catalyst is 0.40 wt. % and the measured Au loading is 0.09 wt. %.

### COMPARATIVE EXAMPLE 2

Preparation of Pd/TS-1 Catalysts

The procedure to make the Pd/TS-1 catalyst is the same as the Catalyst, 1 preparation with the exception that the gold precursor, AuCl₃ is not added to the preparation. Measured Pd loading of the catalyst is 0.41 wt. %.

### COMPARATIVE EXAMPLE 3

Preparation of Au/TS-1 Catalysts

TS-1 (30 g) is dried in vacuum oven at 75° C. then placed in a 1 L glass beaker. Distilled water (400 mL) is added to the beaker and heated to 70° C. on a stirrer-hotplate at medium rpm. Hydrogen tetrachloroaurate (III) trihydrate (HAuCl₄.3H₂O, 0.2524 g) is then added to the distilled water. The pH of the reaction solution is 1.68 and is adjusted to a pH of 7-8 using a 5.0 % NaOH solution. The mixture is stirred for 90 minutes at 70° C., occasionally adding small amounts of the 5% NaOH solution to maintain pH at around 7.5. An additional 600 mL of distilled water is added to the mixture and stirred for 10 minutes. The mixture is then filtered and washed three times with water. Catalyst was dried at 110° C. for 2 hours then calcined at 400° C. for 4 hours. Measured Au loading of the catalyst is 0.2 wt. %.

### EXAMPLE 4

Epoxidation of Propylene Using Catalyst 1 and Comparative Catalysts 2 and 3

To evaluate the performance of the catalysts prepared in Example 1 and Comparative Examples 2 and 3, the epoxidation of propylene using oxygen and hydrogen is carried out. The following procedure is employed.

The catalyst (3 g) is slurried into 100 mL of water and added to the reactor system, consisting of a 300-mL quartz reactor and a 150-mL saturator. The slurry is then heated to 60° C. and stirred at 1000 rpm. A gaseous feed consisting of 10% propylene, 2.5% oxygen, 2.5% hydrogen and 85% nitrogen is added to the system with a total flow of 100 cc/min and a reactor pressure of 3 psig. Both the gas and liquid phase samples are collected and analyzed by G.C.

The epoxidation results, in Table 1, show that the use of a gold promoted Pd/TS-1 catalyst leads to an unexpected improvement in both productivity and selectivity to PO equivalent products (POE=PO, PG, DPG, and acetol) compared to an unpromoted Pd/TS-1 catalyst and Au/TS-1 catalyst.

### COMPARATIVE EXAMPLE 5

Preparation of Pd/TS-1 Catalyst

The TS-1 is calcined at 550° C. for 4 hours before use. PdCl₂ (0.3 g) is dissolved in concentrated NH₄OH (60 g) and water (67 g). The pre-calcined TS-1 (30 g) is added to the palladium solution. After stirring for one hour, the slurry is transferred to a roto-vap and the water is removed by roto-evaporation under vacuum at 80° C. The solid catalyst is then reduced with hydrogen (10% hydrogen in nitrogen) at 100° C. for 3 hours. Measured Pd loading of the catalyst is 0.52 wt. %.

### EXAMPLE 6

Preparation of Pd/Au/TS-1 Catalyst

The unreduced Pd/TS-1 (10 g) from Example 5 is added to a solution of hydrogen tetrachloroaurate (III) trihydrate (0.365 g) in water (21 g). The slurry is stirred for 0.5 hours at room temperature followed by 1.5 hours at 60° C. The slurry is then transferred to a roto-vap and the water is removed by roto-evaporation under vacuum at 80° C. The solid catalyst is then reduced with hydrogen (10% hydrogen in nitrogen) at 100° C. for 3 hours. Measured Pd loading of the catalyst is 0.52 wt. % and the measured Au loading is 1.53 wt. %.

### EXAMPLE 7

Epoxidation of Propylene using Catalyst 6 and Comparative Catalyst 5

To evaluate the performance of the catalysts prepared in Example 6 and Comparative Example 5, the epoxidation of propylene using oxygen and hydrogen was carried out. The following procedure is employed.

The catalyst (3 g) is slurried into 140 mL of water and added to the reactor system, consisting of a 300-mL quartz reactor and a 150-mL saturator. The slurry is then heated to 60° C. at atmospheric pressure. A gaseous feed consisting of 12 cc/min equimolar hydrogen and propylene and 100 cc/min of 5% oxygen in nitrogen is introduced into the quartz reactor via a fine frit. The exit gas is analyzed by on-line GC (PO and ring-opened products in the liquid phase are not analyzed.

The maximum PO observed in the vapor phase (average of 3 one-hour spaced samples) was 1300 ppm PO for Comparative Catalyst 5 and 1600 ppm for Catalyst 6. The ratio of PO produced/O₂ consumed is 15% for Comparative Catalyst 5 and 32% for Catalyst 6. The ratio of PO produced/H₂ consumed is 9% for Comparative Catalyst 5 and 19% for Catalyst 6.

These epoxidation results show that the use of a gold promoted Pd/TS-1 catalyst leads to an unexpected improvement in both productivity and selectivity to PO compared to an unpromoted Pd/TS-1 catalyst.

TABLE 1
Effect of Au Promoter on Catalyst Productivity and Selectivity.

| | Propylene | Oxygen | Hydrogen | PO/RO | POE |
|---|---|---|---|---|---|
| | to POE | to POE | to POE | RO = ring | Productivity |
| | Selectivity | Selectivity | Selectivity | opened | (g POE/g |
| Catalyst | (%) | (%) | (%) | products | cat/h) |
| 1 | 98 | 91 | 90 | 0.25 | 0.017 |
| 2* | 85 | 69 | 40 | 0.63 | 0.0065 |
| 3* | 0.62 | 1.3 | 0.35 | 2.93 | 0.000038 |

*Comparative Example

We claim:

1. A process for producing an epoxide comprising reacting an olefin, hydrogen and oxygen in the presence of a catalyst comprising a titanium zeolite, palladium, and a gold promoter.

2. The process of claim 1 wherein the zeolite is titanium silicalite.

3. The process of claim 1 wherein the zeolite is TS-1.

4. The process of claim 1 wherein the catalyst is comprised of from 0.01 to 5 weight percent palladium.

5. The process of claim 1 wherein the catalyst is comprised of from 0.01 to 2 weight percent gold.

6. The process of claim 1 wherein the olefin is a C₂-C₆ olefin.

7. The process of claim 1 wherein the olefin is propylene.

8. The process of claim 1 further comprising a carrier gas.

9. The process of claim 8 wherein the carrier gas is selected from the group consisting of helium, neon, argon, nitrogen, carbon dioxide, and C₁₋₈ saturated hydrocarbons.

10. The process of claim 8 wherein the carrier gas is propane.

11. The process of claim 1 further comprising a solvent selected from the group consisting of methanol, ethanol, isopropanol, and tert-butanol, and water.

12. A method for preparing a catalyst comprising the steps of:
(a) impregnating a titanium zeolite with a solution of a palladium compound and a gold compound in a solvent;
(b) removing the solvent from the impregnated titanium zeolite; and
(c) drying the impregnated titanium zeolite.

13. The method of claim 12 wherein the titanium zeolite is titanium silicalite.

14. The method of claim 12 wherein the titanium zeolite is TS-1.

15. The method of claim 12 wherein the solvent is water.

16. The method of claim 12 wherein the palladium compound is selected from the group consisting of palladium nitrates, sulfates, halides, carboxylates, and amines.

17. The method of claim 12 wherein the gold compound is selected from the group consisting of gold halides, cyanides, hydroxides, and sulfides.

18. The method of claim 12 wherein the impregnated titanium zeolite is dried at a temperature of from about 50° C. to about 200° C.
